(19) United States
(12) Patent Application Publication
(10) Pub. No.: US 2024/0011079 A1
(43) Pub. Date: Jan. 11, 2024

(54) NUCLEIC ACID DETECTION CHIP AND THE METHOD AND DETECTION EQUIPMENT USING THE SAME

(71) Applicant: NATIONAL APPLIED RESEARCH LABORATORIES, TAIPEI CITY (TW)

(72) Inventors: CHAO-AN JONG, HSINCHU (TW); WANWIPA SIRIWATWECHAKUL, PATHUM THANI (TH); SHU-HAN HSU, HSINCHU (TW); YU-FENG CHENG, TAINAN CITY (TW); PAIBOON SREEARUNOTHAI, PATHUM THANI (TH); THILINA RAJEENDRE KATUGAMPALAGE, PATHUM THANI (TH)

(21) Appl. No.: 17/932,816

(22) Filed: Sep. 16, 2022

(30) Foreign Application Priority Data
Jul. 5, 2022 (TW) 111125227

(51) Int. Cl. C12Q 1/6834; C12Q 1/6816; B01L 7/00

(52) U.S. Cl. CPC C12Q 1/6834; C12Q 1/6816; B01L 7/52; C12Q 2563/143; C12Q 2563/155; C12Q 2563/107; B82Y 15/00

(57) ABSTRACT

The present invention relates to a nucleic acid detection chip, the method and detection equipment using the same. The test sample injects into the first injection hole on the slip plate into the groove on the substrate through the first guide hole. The test sample is heated to the first temperature and then cooled down. Displacing the top plate to align the second injection hole and the hole of the substrate. Injecting the light conversion material into the hole of the substrate to generate a detection sample. Displacing the plate again to move the detection sample to the substrate's top of the detection hole. Exposing the detection sample with the first light to generate the second light by the light conversion material in the detection sample. By absorbing the second light to generate a current that is closely dependent on the concentration of light conversion material in the detection sample.

### FIELD OF THE INVENTION

The invention relates to a method, a device and an apparatus, particularly a nucleic acid detection chip and a method and a detection apparatus thereof.

### BACKGROUND OF THE INVENTION

Novel coronavirus pneumonia is a kind of disease caused by the severe acute respiratory syndrome coronavirus, SARS-CoV-2. It first appeared in Wuhan, China, at the end of 2019. SARS-CoV-2 is an enveloped, positive-strand RNA virus.

SARS-CoV-2 belongs to the beta-coronavirus lineage and belongs to Severe Acute Respiratory Syndrome viruses. The virus attacks through the upper respiratory system, using ACE2 as a viral receptor to infect the body. The main infecting organs are the lungs, heart, and kidney.

The symptoms of infection usually arise in the respiratory system. The clinical manifestations include nasal congestion, runny nose, cough, sore throat, fever, and fatigue. Shortness of breath is seen in about 1 in 3 cases. Other associated symptoms include muscle aches, headaches, and diarrhea; some also experience loss or abnormality of smell or taste. Some infected by the virus present no symptoms.

However, for most, symptoms are usually alleviated in a few weeks. But some patients' conditions deteriorate, causing dyspnea and respiratory distress and requiring intubation and ventilator treatment. This occurs most frequently in senior patients, or patients with pre-existing chronic medical conditions. In the most severe cases, the virus can cause death. Although children can also be infected by the virus, their symptoms are usually milder than adults.

Currently, there are two main forms of testing for COVID-19: “nucleic acid testing” and “antigen rapid testing”. Antigen rapid testing collects specimen from saliva, the nasopharynx, and the nasal cavity to check for viral protein in the specimen to determine whether there is an infection. The test is fast, results are known in about 15 minutes, but is less accurate than nucleic acid testing. As a result, gene sequencing cannot be performed. However, the test procedure is easy and fast, so the test can be used for mass screening at testing stations.

Although the antigen rapid test is fast, and can provide mass screening, it is less accurate than nucleic acid testing and can give false-negative results. Taiwan currently officially uses the more accurate nucleic acid testing.

Nucleic acid testing uses real-time quantitative reverse transcription polymerase chain reaction (real-time RT-qPCR) to detect whether there is SARS-CoV-2 present in the specimen, and the viral load is interpreted using Ct value. The sample is also collected in the nasopharynx and nasal cavity. Collecting specimens and directly detecting genetic material such as viral DNA or RNA has high accuracy, so the nucleic acid test is the current standard for confirming COVID-19 infection or releasing patients from isolation or quarantine at quarantine sites and hospitals.

Even though nucleic acid test is more accurate than the antigen rapid test, the specimen of the test must be sent to a specific laboratory for examination. The relevant experimental equipment for nucleic acid detection is expensive and the technician needs to be trained personnel. Additionally, there is the possibility of cross-contamination. So, despite the accuracy of the test, in light of the severity of the pandemic, many problems with the test still need to be addressed.

The object of the invention is to shorten the nucleic acid test time and to implement a fully automatic programming method to decrease the manpower necessary for the test as well as decrease the possibility of cross-contamination.

### SUMMARY

An object of the invention is to provide a nucleic acid detection chip and the method and detection apparatus thereof. Magnetic nanoparticles are used to adsorb to RNA to shorten test time. In addition, light conversion materials are mixed with the RNA being tested, a light source excites the light conversion materials and produces a light with a specific wavelength, the photoelectric conversion element receives the light and produces a current, and the current is used to determine the concentration of RNA in the sample. The above will increase the accuracy of the test, without expensive high-end instruments, nor highly trained technicians to operate, with no restriction on testing location. This reduces the labor and operation costs.

An object of the invention is to provide a nucleic acid detection chip and the method and apparatus of the chip. The invention uses a unique plate design, control of plate displacement by sliding, and a heating element that extracts nucleic acid from the RNA being tested, then combines and adsorbs the light conversion materials to the RNA being tested and uses a photoelectric element to absorb a light with a specific wavelength to produce a current.

An object of the invention is to provide a nucleic acid detection chip and the method and apparatus, as well as the cleaning device for the apparatus. After testing with the RNA, there is no need for the user to touch the apparatus. Cleaning solution can be used to rinse the RNA out of the groove and, using the configuration of the drainage hole, flush the cleaning fluid containing the RNA into the waste liquid reservoir element. The user does not have to touch the RNA sample, which improves safety and convenience for the user.

An object of the invention is to provide a nucleic acid detection chip and the method and apparatus, as well as the cleaning device thereof. The configuration of the drainage hole allows cleaning solution containing the RNA sample to be flushed into the waste liquid reservoir element. The user does not have to touch the RNA sample, which improves safety and convenience for the user.

In order to achieve the above objectives, the present invention provides a method of testing with the nucleic acid detection chip comprising the steps: inject a test sample into the groove of the substrate via the first injection hole on the plate, and via the first guide hole; use a heating element to heat the substrate and the test sample to the first temperature; cool the test sample to the second temperature; inject magnetic nanoparticles into the groove, via the first guide hole, to combine with and adsorb to the test sample; inject light conversion materials via the first injection hole and the first guide hole into the groove to produce a detection sample; displace the plate laterally so that the test hole is on the groove; use the first magnetic element to attract the detection sample to the light-transmitting groove; displace the plate laterally so that the detection sample in the light-transmitting groove is on the hole in the substrate. Irradiate the detection sample, via the hole, by the first light of the light source and use the light conversion materials in the detection sample to convert the first light to the second light; absorb the second light by the photoelectric conversion element and produce a current. The first temperature is over 50° C. and under 100° C. . The second temperature is between 20° C. and 30° C. The wavelength of the light is from 300 nm to 700 nm.

The invention provides an embodiment of a detection method on the nucleic acid detection chip, wherein the test sample contains a specimen and a chemical that is the protein kinase.

The invention provides an embodiment of a detection method on a nucleic acid detection chip, wherein after the procedure of adsorbing the magnetic nanoparticles to the test sample comprise the steps: use the second magnetic element to attract the test sample on which magnetic nanoparticles are adsorbed to the bottom of the groove.

The invention provides an embodiment of a detection method for the nucleic acid detection chip, wherein in the step of using a second magnetic element to attract the test sample on which magnetic nanoparticles are adsorbed to the bottom of the groove, comprising the steps: continuously inject cleaning solution such that it flows via the first guide hole into the groove to remove the test sample that does not have magnetic nanoparticles adsorbed and the cleaning solution flows via the first drainage hole on the side of the first guide hole to the waste liquid reservoir element. The cleaning solution is ethanol or acetone.

The invention provides an embodiment of a detection method for the nucleic acid detection chip, wherein in the step of injecting light conversion materials via the first injection hole and first guide hole into the groove and producing a detection sample comprises the steps: continuously inject cleaning solution such that it flows via the first guide hole into the groove to remove light conversion materials not adsorbed to the test sample and the cleaning solution flows via the first drainage hole on the side of the first guide hole to the waste liquid reservoir element. The cleaning solution is ethanol or acetone.

The invention provides an embodiment of a detection method for the nucleic acid detection chip, wherein the light conversion materials are fluorescein, phosphor, or quantum dots.

In order to achieve the above objectives, the present invention provides a configuration of the nucleic acid detection chip comprising: a sliding chip element, which has a plate and a substrate. The substrate includes the first body, a groove, and a hole. The plate includes the second body, the first injection hole, and a light-transmitting groove. The plate slide is on the substrate, the groove is on the top of the first body, the first injection hole is connected to the first guide hole, the light transmitting groove is on the side of the first injection hole, and the photoelectric conversion element is on the top of the light-transmitting groove. Wherein the groove contains a detection sample, which has the test sample with magnetic nanoparticles and light conversion materials adsorbed on. Through sliding, the plate causes the light-transmitting groove to be above the groove and attracts the detection sample to the light-transmitting groove by the first magnetic element. Through sliding, the plate causes the light-transmitting groove to be displaced above the hole and allows the first light of the light source to irradiate the detection sample. The light conversion materials convert the first light to the second light, and the photoelectric element absorbs the second light and produces a current.

The invention provides an embodiment of the configuration of the nucleic acid detection chip, wherein the test sample is injected into the groove via the first injection hole and first guide hole; the substrate is heated by a heating element to the first temperature and cooled to the second temperature; the magnetic nanoparticles are injected via the first injection hole and the first guide hole to the groove and adsorbed to the test sample; the light conversion materials are injected into the groove via the first injection hole and the first guide hole, and adsorbed to the test sample to produce a detection sample. The test sample includes a specimen and a chemical that is the protein kinase. The first temperature is over 50° C. and under 100° C. The second temperature is between 20° C. and 30° C.

The invention provides an embodiment of the configuration of the nucleic acid detection chip, wherein a cooling line containing coolant surrounds the groove, a light filter element is above the light-transmitting groove, and a light-transmitting element is above the light filter element.

The invention provides an embodiment of the configuration of the nucleic acid detection chip, wherein the wavelength of the light is from 200 nm to 700 nm, and the light conversion materials are fluorescein, phosphor, or quantum dots, which are used to convert the wavelength of the light.

The invention provides an embodiment of the configuration of the nucleic acid detection chip, wherein the first injection hole has the first diameter, and the first guide hole has the second diameter. The first diameter is bigger than the second diameter.

Additionally, the invention provides a detection apparatus for the nucleic acid detection chip, comprising a carry base, on which there is a light source, a sliding chip element on the carry base that has a substrate that has the first body, a groove, and a hole, and a plate that has the second body, the first injection hole, and the light-transmitting groove; the plate slide is on the substrate, the groove is on the first body, the first injection hole is connected to the first guide hole, and the light-transmitting groove is on the side of the first injection hole. The photoelectric conversion element that is on the light-transmitting groove, a moving element that includes a moving part and an electrode sensing part, and a display element that is electrically connected to the electrode sensing part. The moving part is on the side of the plate, and the electrode sensing part is electrically connected to the photoelectric conversion element. Wherein the groove contains a detection sample, which has the test sample with magnetic nanoparticles and light-conversion materials adsorbed on. Through sliding the plate by the moving part, the light-transmitting groove is displaced on the groove and attracts the detection sample to the light-transmitting groove with the first magnetic element. Through sliding the plate, the light-transmitting groove is displaced on the hole and allows the first light of the light source to irradiate the detection sample. The light conversion material converts the first light to the second light, and the photoelectric element absorbs the second light and produces a current, which is received by the electrode sensing part; the result from the test is then reflected on the display element.

Additionally, the invention provides a cleaning method and device for the nucleic acid detection chip, comprising: inject a test sample into the groove of the substrate via the first injection hole on the plate, and via the first guide hole; inject magnetic nanoparticles into the groove, via the first guide hole, to combine with and adsorb to the test sample; inject light conversion materials via the first injection hole and the first guide hole into the groove to produce a detection sample; displace the plate laterally so that the test hole is on the groove; use the first magnetic element to attract the detection sample to the light-transmitting groove; displace the plate laterally so that the detection sample in the light-transmitting groove is on the hole in the substrate. Irradiate the detection sample, via the hole, by the first light of the light source and use the light conversion materials in the detection sample to convert the first light to the second light; absorb the second light by the photoelectric conversion element and produce a current. The steps in the method for the nucleic acid detection chip's cleaning device comprise: displace the plate laterally so that the light-transmitting groove on the plate is on the groove of the substrate. Inject cleaning solution via the cleaning channel on the side of the light-transmitting groove into the groove to remove the detection sample in the light-transmitting groove and the groove. The cleaning solution will flow via the second drainage hole on the side of the light-transmitting groove to the waste liquid reservoir element. Additionally, the invention provides a cleaning device for the nucleic acid detection chip, comprising a substrate that has the first body, a groove, and a hole, with the groove on the body; a plate that has the second body with the first and second injection holes on the side. The first injection hole is connected to the first guide hole and the second injection hole is connected to a light-transmitting groove. The first injection hole and second injection hole have the first diameter, and the first guide hole and light-transmitting groove have the second diameter. The first diameter is bigger than the second diameter. There is a drainage hole between the first guide hole and the light-transmitting groove. The photoelectric conversion element is on the top of the second injection hole. The plate is displaced laterally so that the second injection hole is above the groove on the substrate, and cleaning solution can be injected into the second injection hole. The cleaning solution flows via the light-transmitting groove into the groove to remove the detection sample in the light-transmitting groove and groove. The cleaning solution flows via the drainage hole on the side of the light-transmitting groove to the waste liquid reservoir element.

The invention provides an embodiment for a cleaning device for the nucleic acid detection chip, comprising a heating element on the side of the substrate that heats the substrate to the first temperature. The first temperature is over 50° C. and under 100° C.

The invention provides an embodiment for a cleaning device for the nucleic acid detection chip, comprising a cooling line containing coolant that surrounds the groove, a light filter element above the light-transmitting groove, and a light-transmitting element above the light filter element.

The invention provides an embodiment for a cleaning device for the nucleic acid detection chip, wherein the first injection hole has the first diameter, and the first guide hole has the second diameter. The first diameter is bigger than the second diameter.

### BRIEF DESCRIPTION OF THE FIGURES

FIG. 1A is a flow chart of the steps of the first embodiment of the invention

FIG. 1B is a structural drawing of the first embodiment of the invention;

FIG. 1C is an enlarged structural drawing of the first embodiment of the invention;

FIG. 2A-2H: They are diagrams of the use conditions of the first embodiment of the invention;

FIG. 3A is a flow chart of the steps of the second embodiment of the invention;

FIG. 3B is a diagram of the use conditions of the second embodiment of the invention;

FIG. 4A is a flow chart of the steps of the third embodiment of the invention;

FIG. 4B is a flow chart of the use conditions of the third embodiment of the invention;

FIG. 5A is a flow chart of the steps of the fourth embodiment of the invention;

FIG. 5B is a flow chart of the use conditions of the fourth embodiment of the invention;

FIG. 6A is a flow chart of the steps of the fifth embodiment of the invention;

FIG. 6B is a flow chart of the use conditions of the fifth embodiment of the invention;

FIG. 7A is a flow chart of the steps of the sixth embodiment of the invention; and

FIG. 7B is a flow chart of the use conditions of the sixth embodiment of the invention.

### DETAILED DESCRIPTION

Nucleic acid testing requires specimens to be sent to specific laboratories and a lengthy process of examination to obtain a result. In face of the severity of the pandemic, the nucleic acid test must be improved to address the problem of a large number of cases and patients

To address this problem, the invention provides a nucleic acid detection chip, a method, and a detection apparatus thereof. Magnetic nanoparticles are used to adsorb to RNA to shorten test time. In addition, light conversion materials are mixed with the RNA being tested, a light source excites the light conversion materials and produces a light with a specific wavelength, the photoelectric conversion element receives the light and produces a current, and the current is used to determine the concentration of RNA in the sample. The above will increase the accuracy of the test, without expensive high-end instruments, nor highly trained technicians to operate, with no restriction on testing location. This reduces the labor and operation costs.

After testing with the RNA, there is no need for the user to touch the apparatus. Cleaning solution can be used to rinse the RNA out of the groove and, using the configuration of the drainage hole, flush the cleaning fluid containing the RNA into the waste liquid reservoir element. The user does not have to touch the RNA sample, which improves safety and convenience for the user.

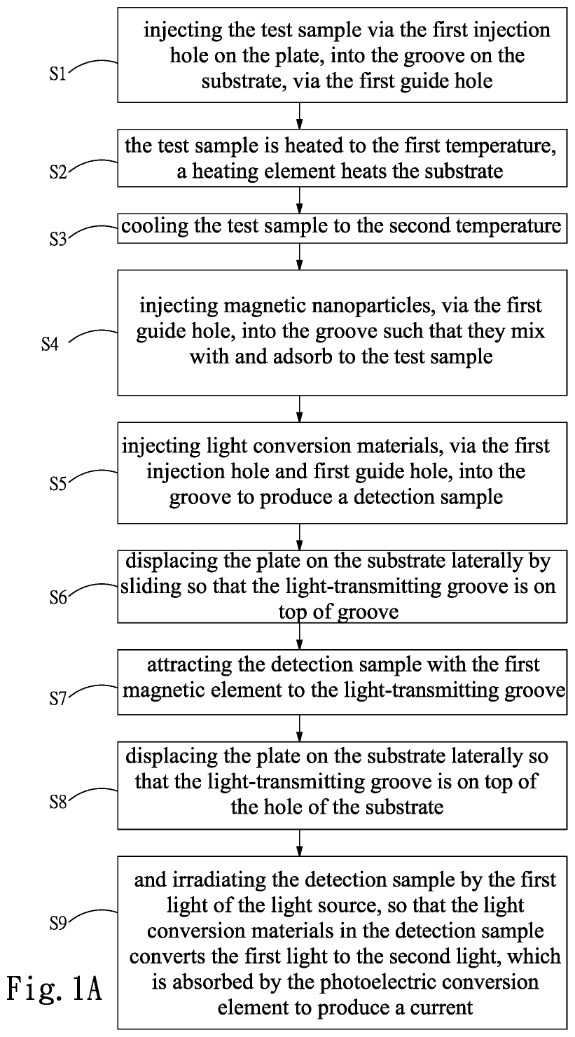

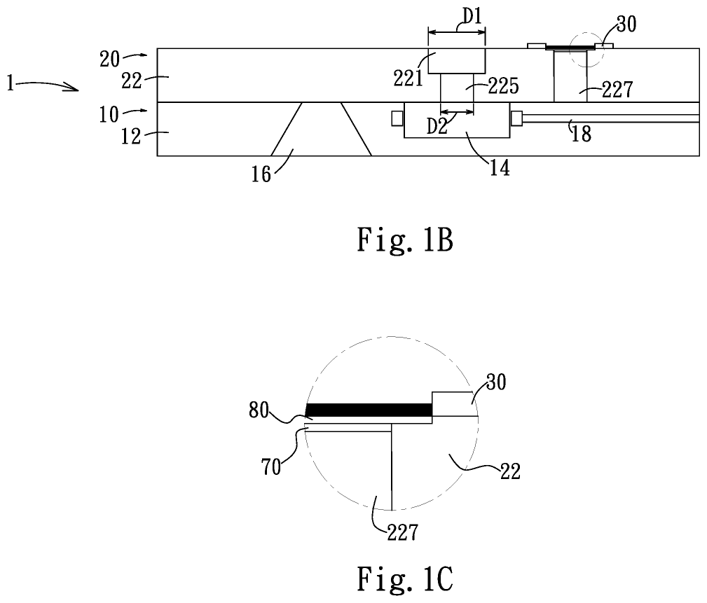

Refer to FIG. 1A, which is the flow chart of the steps of the first embodiment of the invention, and FIG. 1B, which is a structural drawing of the first embodiment of the invention. As shown in FIG. 1A, the steps in the method for the nucleic acid detection chip comprise:
Step S1: Inject a test sample into the groove of the substrate via the first injection hole on the plate, and via the first guide hole.
Step S2: Use a heating element to heat the test sample to the first temperature.
Step S3: Cool the test sample to the second temperature.
Step S4: Inject magnetic nanoparticles into the groove, via the first guide hole, to combine with and adsorb to the test sample.
Step S5: Inject light conversion materials via the first injection hole and the first guide hole into the groove to produce a detection sample.
Step S6: Displace the plate laterally so that the light-transmitting groove on the plate is on the groove.
Step S7: Use the first magnetic element to attract the detection sample to the light-transmitting groove
Step S8: displace the plate laterally so that the detection sample in the light-transmitting groove is on the hole in the substrate.
Step S9: Irradiate the detection sample, via the hole, by the first light of the light source and use the light conversion materials in the detection sample to convert the first light to the second light, and absorb the second light by the photoelectric conversion element to produce a current.

This embodiment contains a configuration of the nucleic acid detection chip, as shown in FIG. 1B, which includes first sliding chip element 1. First sliding chip element includes substrate 10 and plate 20. The substrate 10 includes the first body 12, groove 14, and hole 16. The groove 14 is on the top of the first body 12 and is beside the hole 16.

In the embodiment, the plate 20 includes second body 22, first injection hole 221 and light-transmitting groove 227 are provided on one side of second body 22, and the first injection hole 221 connected to first guide hole 225, light-transmitting groove 227 is on the side of first guide hole 225, first injection hole 221 has a first diameter D1, and first guide hole 225 has a second diameter D2, the first diameter D1 is larger than the second diameter D2.

Refer to FIG. 1C, which is an enlarged structural drawing of the first embodiment of the invention. As shown in the figure, light filter element 70 is above light-transmitting groove 227, and light-transmitting element 80 is above the light filter element 70.

In the embodiment, photoelectric conversion element 30 is on the top of light-transmitting groove 227.

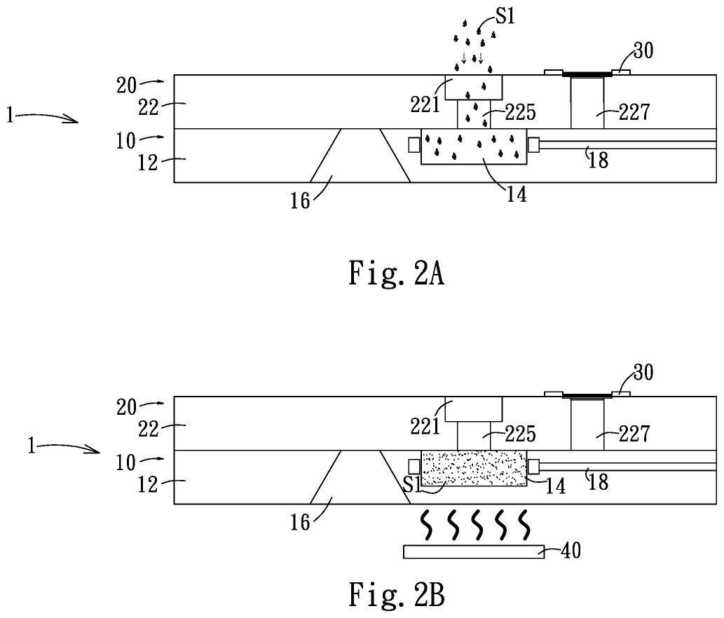

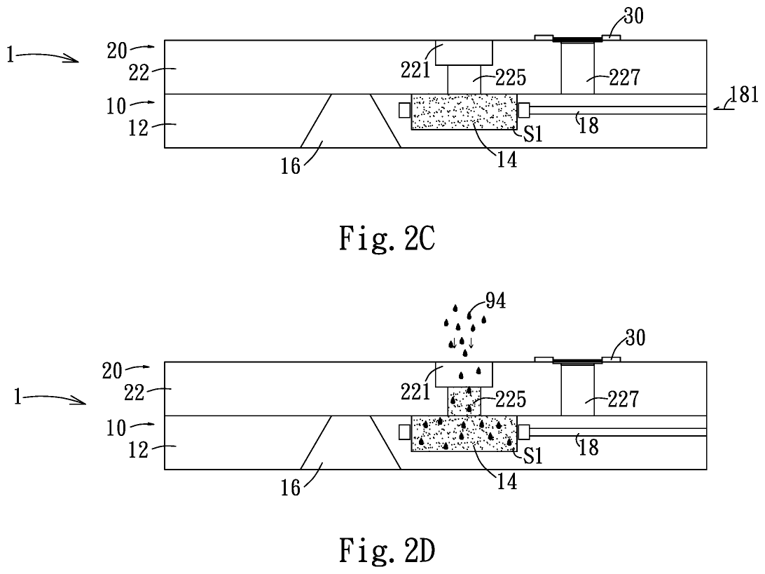

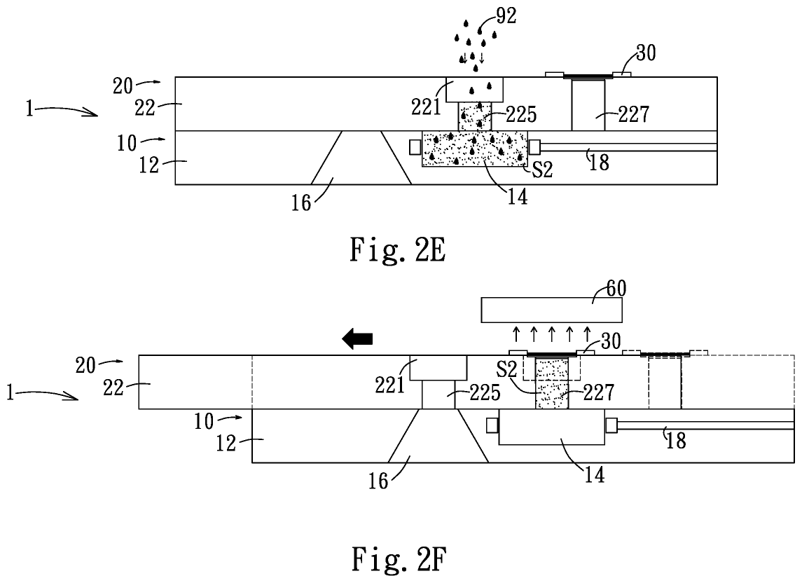

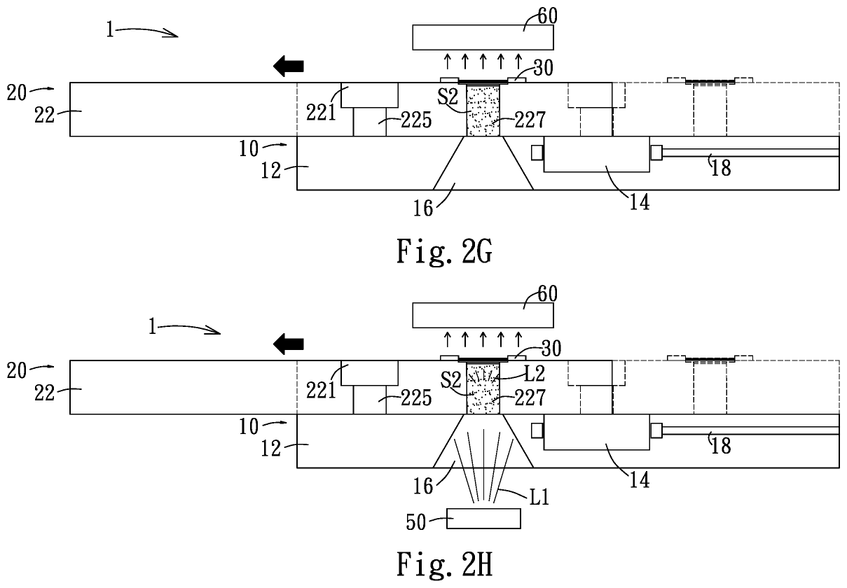

Further, refer to FIG. 2A-2H, which are diagrams of the use conditions of the first embodiment of the invention, as shown in FIGS. 2A-2H and step S1, test sample S1 is injected into groove 14 on substrate 10 via first injection hole 221 on plate body 20 and first guide hole 225 (as shown in FIG. 2A), wherein the test sample contains a specimen and a chemical; the specimen above is the saliva of a test subject, and the chemical is the protein kinase.

Wherein protein kinase is responsible for the phosphorylation of proteins in the human body, and it has a relative function with the protein phosphatases responsible for the dephosphorylation of proteins. The phosphorylation of proteins determines the structure and activity of proteins and also affects the process of intracellular message transmission. The human genome contains about 500 protein kinase genes, accounting for about 2% of human genes.

Additionally, the test sample S1 can also be an antibody, an antigen, a deoxyribonucleic acid (DNA) probe, a ribonucleic acid (RNA) probe, an enzyme, a protein, a globulin or at least one biologically active composition. Prior to covalent bonding, at least one biologically active functional group is selected from hydroxyl, alkyl, amine, carboxylic acid, ester, thioester, aldehyde, epoxy, ethoxy, ethane, oxirane group, hydrazine group or thiol group. Prior to non-covalent bonding, at least one biologically active functional group is selected from a group consisting of biotin, avidin, streptavidin, protein, deoxyribonucleic acid (DNA), ribonucleic acid (RNA), ligand or receptor, but not limited thereto.

In the embodiment, as shown in step S2, heating element 40 is used to heat substrate 10 and test sample S1 to the first temperature (as shown in FIG. 2B), wherein the first temperature is over 50° C. and under 100° C.

Further, in the embodiment, as shown in step S3, after heating the test sample S1 to the first temperature it is cooled to a second temperature, wherein the first temperature is between 20° C. to 30° C., and the test sample S1 is cooled by coolant 181 in cooling line 18 surrounding groove 14.

In the embodiment, as shown in steps S4 to S5, after the test sample S1 is cooled to the second temperature (as shown in FIG. 2C), magnetic nanoparticles are injected into groove 14 via first guide hole 221 to combine and adsorb with test sample S1 (as shown in FIG. 2D), and light conversion materials 92 are injected into groove 14 via first injection hole 221 and first guide hole 225 to produce detection sample S2 (as shown in FIG. 2E).

Wherein, light conversion materials 92 are fluorescein, phosphor, or quantum dots, wherein fluorescein is a kind of synthetic organic compound, and the appearance is a dark orange or red powder, soluble in ethanol and slightly soluble in water. Under blue light or ultraviolet light, it will emit green fluorescence. The structure of fluorescein is as follows:

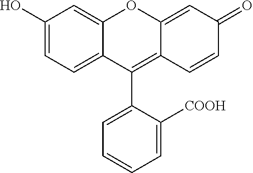 [SMILES: O=C(O)c1ccccc1-c1c2ccc(=O)cc-2oc2cc(O)ccc12]

It is widely used as a fluorescent tracer in a variety of technical applications (such as fluorescent antibody technology). For example, in diseases of ocular surface in the clinical, sodium fluorescein is used as stained material, observed under blue light and used to assess whether the corneal barrier function is damaged.

Phosphors use magnesium tungstate, calcium tungstate, zinc silicate, calcium halophosphate, lanthanum oxyfluoride phosphors or rare earth phosphors (such as yttrium oxide, lanthanum oxide), but are not limited to the aforementioned ingredients. Phosphor is also commonly known as luminous powder, which is usually divided into two categories: photo-induced energy storage luminous powder and noctilucent luminous powder. Photo-induced energy storage luminous powder is a fluorescent powder that stores light energy after being irradiated by natural light, fluorescent light, ultraviolet light, etc., and then slowly releases it in the form of fluorescent light after the light irradiation is stopped. In the dark, the glow can still be seen, lasting for a few hours to a couple dozens of hours.

Additionally, a quantum dot (Quantum Dot, QD) is a nanocrystal (Nanocrystal) semiconductor material, which is composed of II-VI, III-V or IV-VI group elements. Different from the bulk semiconductors, the grain diameter of quantum dots is only about 2.0 nm to 10 nm, which is equivalent to the width of 10˜50 atoms. For example, if the diameter of a quantum dot is 5 nm, the total width of 4 million quantum dots connected in series is about 20 mm, which is equivalent to the diameter of a coin. It can be seen that it is a very tiny particle. Common components of quantum dots are CdSe, ZnS, PbS or InP, but are not limited to the aforementioned components.

In this embodiment, after detection sample S2 is generated, as shown in step S6, plate 20 is displaced laterally, so that light-transmitting groove 227 on the plate 20 is on groove 14 (as in FIG. 2F).

In this embodiment, as shown in steps S7 to S8, detection sample S2 is attracted to light-transmitting groove 227 through first magnetic element 60, and plate 20 is displaced laterally so that detection sample S2 in light-transmitting groove 227, which is on plate 20, is on hole 16 in substrate 10 (as shown in FIG. 2G).

Finally, in this embodiment, as shown in step S9, a first light L1 of a light source 50 irradiates detection sample S2 via hole 16 and light-transmitting groove 227. Detection sample S2 receives the first light L1 and uses light conversion materials 92 in detection sample S2 to convert the first light L1 into light L2, which is then absorbed by photoelectric conversion element 30 to produce a current (not shown). The wavelength of light 50 in this embodiment is from 300 nm to 700 nm (as shown in FIG. 2H).

Wherein, light filter element 70 filters the first light L1 not converted in detection sample S2, while the second light L2 passes through filter element 70 and light-transmitting element 80 at the same time to be absorbed and converted by photoelectric conversion element 30 to generate a current.

A key part in nucleic acid detection is photoelectric conversion element 30; it is a device that converts a fluorescent signal into an electrical signal based on the photoelectric effect. Components commonly used in fluorescent signal detection include photomultiplier tube (Photomultiplier tube, PMT), photodiode (Photodiode, PD) and charge coupled device (Charge coupled device, CCD).

A photomultiplier tube (PMT), mentioned above, is a vacuum electronic device that can convert weak fluorescent signals into electrical signals for amplification. The principle is as follows: when the photocathode receives the fluorescent signal, it emits photoelectrons into the vacuum and enters multiple multiplication systems in series. The multiplied electrons are collected from the anode and outputted in the form of photocurrent.

A photodiode (PD), mentioned above, is a semiconductor device that can convert a fluorescent signal into an electrical signal. It is also a photodetector that can convert light into a current or a voltage signal according to the usage mode.

Although a photodiode, compared to a photomultiplier tube, has a smaller photosensitive area, it has better linearity and lower signal noise rate (S/N), is small and affordable. So, they are used more and more often in fluorescent lamps and light detection instruments, especially in portable devices.

A charge-coupled device (CCD), mentioned above, is an integrated circuit with many capacitors arranged neatly on it, which can detect light and convert images into digital signals.

An example of use of the embodiment is described as follows:

When the suspected infected person (hereinafter referred to as A) seeks rapid testing, ask A to spit out saliva (the specimen) into groove 14, and then react with chemicals in groove 14 (the test sample S1 is formed here), test sample S1 is heated to 75° C. (the first temperature) by heating element 40, and then cooled to 25° C. (the second temperature). Magnetic nanoparticles 94 are injected into groove 14 via first guide hole 225 of plate 20, which is above groove 14, and magnetic nanoparticles 94 are combined with and adsorb to test sample S1. The quantum dots (light conversion materials 92) are injected into groove 14 via first injection hole 221 and first guide hole 225 to produce detection sample S2. Plate 20 is displaced laterally, so that light-transmitting groove 227 on plate 20 is on groove 14.

Then, first magnetic element 60 attracts, magnetically, detection sample S2 containing magnetic nanoparticles 94 (and also containing light conversion materials 92) to light-transmitting groove 227. Plate 20 is displaced laterally again, so that detection sample S2 in the light-transmitting groove 227 is on hole 16 in substrate 10.

Then, the first light L1, generated by turning on light 50 irradiates detection sample S2 via hole 16; the first light L1 passes through and is absorbed by detection sample S2. Light conversion materials 92 convert the first light L1 into the second light L2, and after photoelectric conversion element 30 absorbs the second light L2, a current is produced. Different currents corresponding to the concentration of test sample S1 will be produced, and the results can be used by professionals to determine whether there is infection.

The first embodiment of the invention has the advantage of combing light conversion materials with the RNA being tested, exciting the light conversion material by the light source to produce a light with a specific wavelength, and the photoelectric conversion element receives the light with the specific wavelength to generate a current, and the current is used to determine the concentration of RNA in the sample. This method will increase the accuracy of the test, without expensive high-end instruments, nor highly trained technicians to operate, with no restriction on testing location. This reduces the labor costs and operating costs.

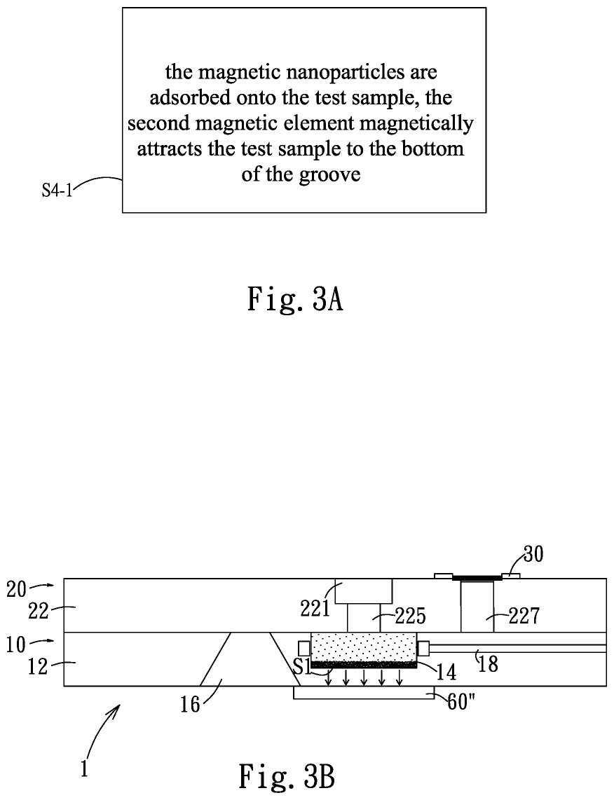

Further, refer to FIG. 3A, which is a flow chart of the steps of the second embodiment of the invention. As shown in the figure, steps S1 to S9 are the same as those of the first embodiment, and will not be repeated here. But in step S4, it further includes steps:
Step S4-1: Use the second magnetic element to attract the test sample on which magnetic nanoparticles are adsorbed to the bottom of the groove.

With FIG. 3B, which is a diagram of the use conditions of the second embodiment of the invention. When proceeding to step S4-1, in step S4 (as shown in FIG. 2D), use second magnetic element 60 to attract test sample S1 on which magnetic nanoparticles are adsorbed to the bottom of groove 14 (as shown in FIG. 3B).

An example of use of the embodiment is described as follows:

When the suspected infected person (hereinafter referred to as A) seeks rapid testing, ask A to spit out saliva (the specimen) into groove 14, and then react with chemicals in groove 14 (the test sample S1 is formed here), test sample S1 is heated to 75° C. (the first temperature) by heating element 40, and then cooled to 25° C. (the second temperature). Adsorb magnetic nanoparticles 94 to test sample S1 and use second magnetic element 60 to attract test sample S1 to the bottom of groove 14. This separates test sample S1 that has magnetic nanoparticles 94 adsorbed, and test sample S1 that does not have magnetic nanoparticles 94 adsorbed; this will help subsequent detection.

Through the application of the magnetic nanoparticles, the effect that the second embodiment of the invention can achieve is that the magnetic nanoparticles 94 can be used to quickly detect the target through magnetic attractive force. The detection targets can be cells, bacteria, DNA or RNA. The special advantages of separation technology by magnetic attractive force are rapid screening, easy operation, not requiring a centrifuge, and ability to handle lots of samples. This reduces time and manpower necessary.

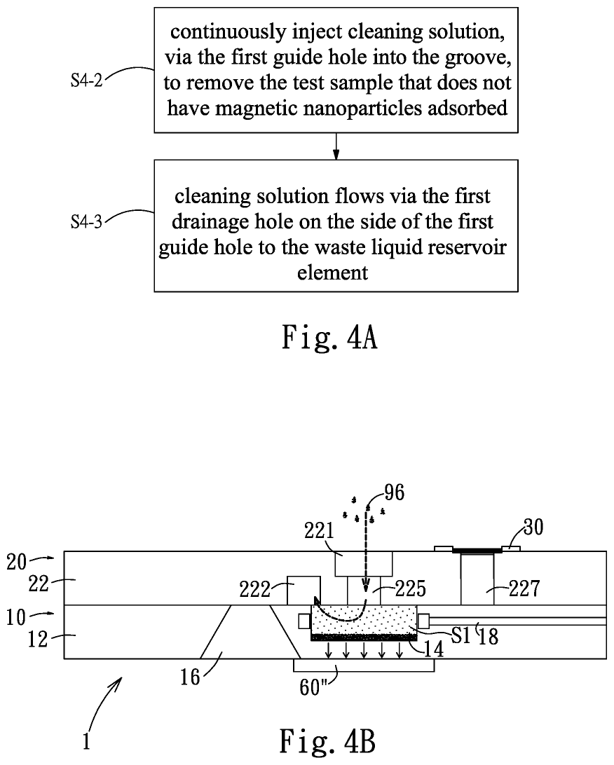

Also, refer to FIG. 4A, which is a flow chart of the steps of the third embodiment of the present invention. As mentioned above in step S4-1, the invention further comprises:
Step S4-2: Continue to inject the cleaning solution, so that the cleaning solution continues to flow via the first guide hole into the groove, so as to remove the magnetic nanoparticles test sample that does not adsorb to the test sample; and
Step S4-3: The cleaning solution is guided to the waste liquid reservoir element for storage via the first drainage hole on the side of the first drainage hole.

In this embodiment, as shown in steps S4-2 and S4-3, refer to FIG. 4B, which is a flow chart of the use conditions of the third embodiment of the invention. As shown in the figure, cleaning solution 96 is continuously injected into groove 14 via first guide hole 221 to remove the magnetic nanoparticles 94 that do not adsorb to test sample S1 (as shown in FIG. 4B). Cleaning solution 96 will be guided to a waste liquid reservoir element (not shown) via first drainage hole 222 on the side of first guide hole 221, wherein cleaning solution 96 is ethanol or acetone.

An example of use of the embodiment is described as follows:

After second magnetic element 60 attracts test sample S1 on which magnetic nanoparticles 94 are adsorbed to the bottom of the groove 14, cleaning solution 96 continuously flows into the first guide hole 221, removing test sample S1 that does not have magnetic nanoparticles 94 adsorbed on, so as to avoid the possibility of misjudgment during subsequent detection.

By the application of cleaning solution 96, the third embodiment of the present invention can achieve the effect of removing test sample S1 that does not have magnetic nanoparticles 94 adsorbed on. There is no need for the user to touch test sample S1, which reduces the risk of infection, as well as the possibility of misjudging the result due to cross-contamination.

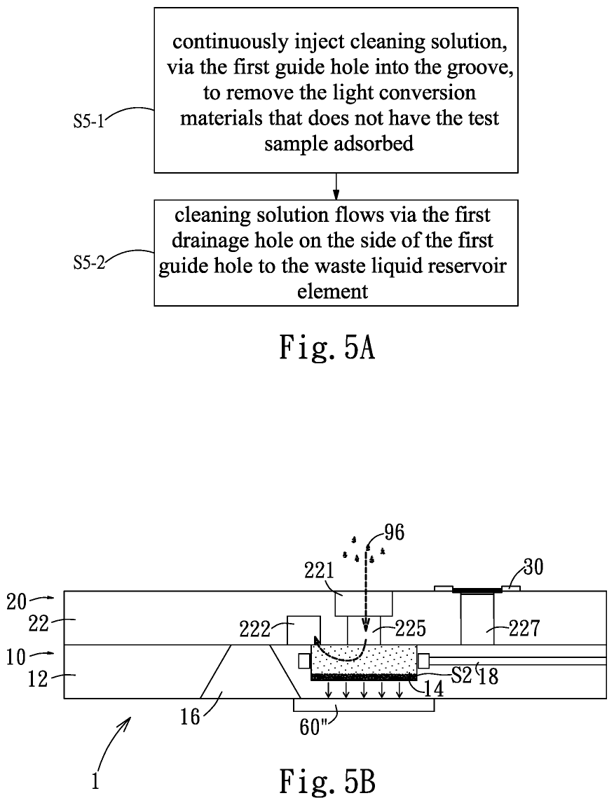

Also, refer to FIG. 5A, which is a flow chart of the steps of the fourth embodiment of the invention. As shown in the figure, steps S1 to S9 are the same as those of the first embodiment and will not be repeated here. S5, which repeats step S4-1, and further includes steps:
Step S5-1: Continue to inject cleaning solution, so that the cleaning solution flows into the groove, via the first guide hole, so as to remove light conversion materials that do not adsorb test sample; and
Step S5-2: The cleaning solution is guided to the waste liquid reservoir element for storage via the drainage hole on the side of the first guide hole.

In this embodiment, as shown in steps S5-1 and S5-2, second magnetic element 60 is used to magnetically attract test sample S1, which contains light conversion materials 92 (as well as magnetic nanoparticles 94, which is also in the detection sample S2 referred to in step S5). Cleaning solution is continuously injected into groove 14 via first guide hole 225 to remove light conversion materials 92 that are not adsorbed to test sample S1. Cleaning solution 96 is then guided to the waste liquid reservoir element for storage via the second drainage hole 224 on the side of first guiding hole 225, wherein the cleaning solution 96 is ethanol or acetone.

An example of use of the embodiment is described as follows:

When light conversion materials 92 adsorb to test sample S1 to form detection sample S2, cleaning solution 96 is continuously injected into light-transmitting groove 227 to remove light conversion materials 92 that are not adsorbed to detection sample S2. This avoids misjudging the light from the fluorescent emission during subsequent detection, so that the subsequent detection result is correct.

By application of cleaning solution 96, the fourth embodiment of the present invention can achieve the effect of directly washing light conversion materials 92 that do not adsorb to detection sample S2, without need for the user to touch detection sample S2, which reduces the risk of infection, and risk of misjudging the result due to contamination of incomplete cleaning.

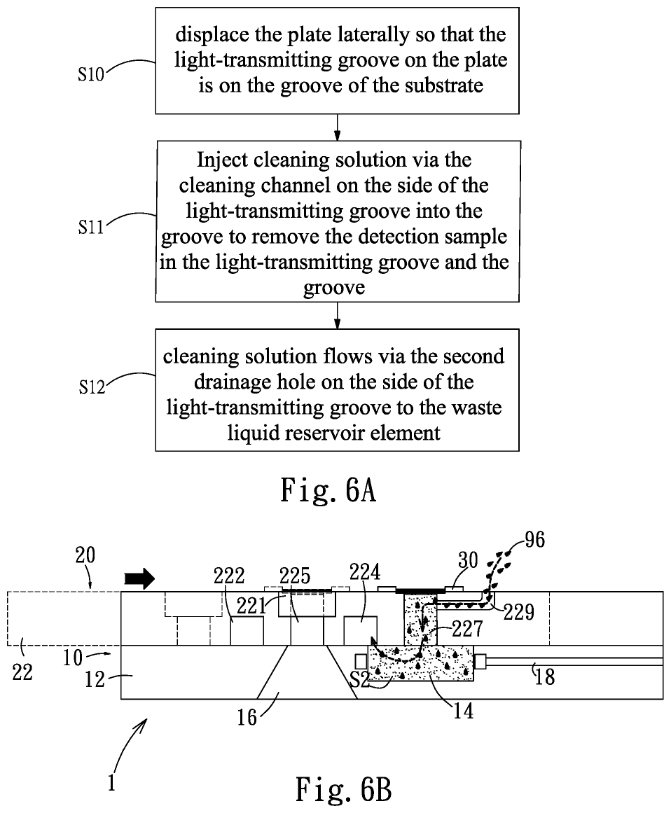

Furthermore, the configuration of the nucleic acid detection chip can be more convenient, fast and concise in use. The present invention further provides a cleaning method and apparatus thereof. Refer to FIG. 6A, which is a flow chart of steps of the fifth embodiment of the invention, and FIG. 6B, which is a flow chart of the use conditions of the fifth embodiment of the invention.

In this embodiment, as shown in FIG. 6A, there is a cleaning method of the cleaning device of a nucleic acid detection chip, and FIG. 6B, which is a flow chart of the use conditions of the fifth embodiment of the invention. After the first, second, third, and fourth embodiments, the steps of the cleaning method comprise:
Step S10: laterally displace the plate, so that the light-transmitting groove on the plate is on the groove of the substrate;
Step S11: inject a cleaning solution into the cleaning channel on the side of the light-transmitting groove, so that the cleaning solution is injected continuously via the light-transmitting groove into the groove, then remove the test sample from the light-transmitting groove and the groove; and
Step S12: The cleaning solution is guided to the waste liquid reservoir element for storage via the second drainage hole on THE side of the light-transmitting groove.

For this embodiment, the configuration and connection of the cleaning device of the nucleic acid detection chip are the same as those of the first embodiment, so they will not be repeated here. A second drainage hole 224 is included.

In this embodiment, plate 20 is displaced laterally, so that light-transmitting groove 227 on plate 20 is on groove 14 of substrate 10. Cleaning solution 96 is injected into cleaning channel 229 on the side of light-transmitting groove 227, so cleaning solution 96 flows via cleaning channel 229 to light-transmitting groove 227, then into groove 14 to remove detection sample S2 from light-transmitting groove 227 and groove 14. Cleaning solution 96 is guided to a waste liquid reservoir element (not shown) via second drainage hole 224 on the side of light-transmitting groove 227 for storage, wherein first injection hole 221 has a first diameter D1 and first guide hole 225 has a second diameter D2, and the first diameter D1 is larger than the second diameter D2.

And in this embodiment, there is heating element 40, which is on the side of the substrate. Heating element 40 heats substrate 10 to the first temperature, the first temperature over 50° C. and under 100° C.

Further, this embodiment further comprises cooling line 18, filter element 70 and light-transmitting element 80, wherein groove 14 is surrounded by cooling line 18, and cooling line 18 carries coolant 181; filter element 70 is on light-transmitting groove 227, and light-transmitting element 80 is on filter element 70.

In this embodiment, by the application of cleaning solution 96 and second drainage hole 224, the effect that the fifth embodiment of the invention can achieve is, using the configuration of the drainage hole, the RNA containing the cleaning solution is guided to the waste liquid reservoir element for storage, and the user does not need to touch the RNA sample, which improves safety and convenience for the user. Additionally, when cleaning channel 229 is not in use, a valve (not shown in the figure) can be provided at one end of cleaning channel 229 to control the valve element to open and/or close the cleaning channel, then to control the volume of cleaning solution 96.

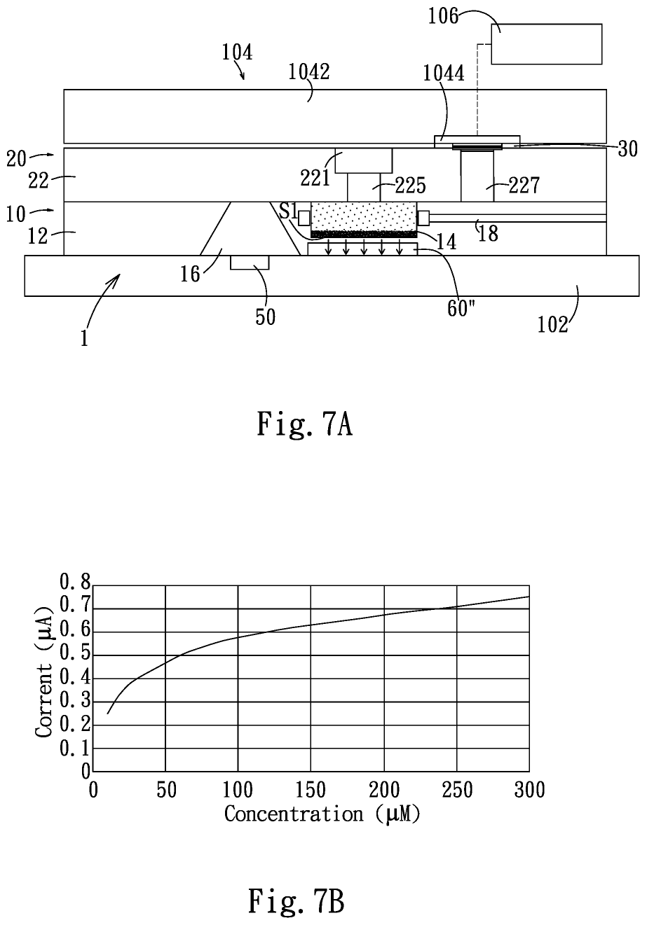

In addition, refer to FIG. 7A, which is a flow chart of the steps of the sixth embodiment of the invention. In this embodiment, a nucleic acid detection chip detection apparatus, wherein substrate plate 20 and photoelectric conversion element 30 of sliding chip element 1 are the same as those in the first embodiment, so they will not be repeated here. In this embodiment, carry base 102 is further on which there is light 50, moving element 104 which includes moving part 1042 and electrode sensing part 1044, moving part 1042 is on the side of plate 20; electrode sensing part 1044 is electrically connected to photoelectric conversion element 30. In the embodiment, display element 106 is included, which is electrically connected to electrode sensing element 1044.

Wherein, in this embodiment, groove 14 contains detection sample S2, which is irradiated by the first light L1 of light source. First light L1 passes through light conversion material 92 within detection sample S2, and then is converted into second light L2. Photoelectric conversion element 30 absorbs the second light L2 and produces a current, and the electrode sensing element 1044 receives the current. As shown in FIG. 7B, which is a flow chart of the use conditions of the sixth embodiment of the invention, the detection result from the test is reflected on display element 106.

As shown in FIG. 7B, the result is reflected on display element 106, wherein the X-axis is the concentration, the Y-axis is the current. Through a calibration curve to check the result reading, it can be used to determine whether detection sample S2 contains the detection target. The concentration range of the test sample can be judged from 10 micromoles/microliter to 50 nanomoles/microliter.

Further, as mentioned in the above embodiments, first guide hole 225 can be many holes, so that test sample S1, the detection sample S2, light conversion materials 92, magnetic nanoparticles 94 and cleaning solution 96 can be injected into groove 14 separately to avoid cross-reaction in first guide hole 225 too soon.

What is claimed is:

1. A method of testing with the nucleic acid detection chip, comprising:
injecting a test sample via a first injection hole on the plate, and sending the test sample into the groove on the substrate via the first guide hole;
heating the test sample to the first temperature;
cooling the test sample to the second temperature;
injecting a magnetic nanoparticles, via the first guide hole, into the groove such that they mix with and adsorb to the test sample;
injecting a light conversion materials, via the first injection hole and the first guide hole, into the groove to produce a detection sample;
displacing the plate on the substrate laterally by sliding so that a light-transmitting groove is on top of groove;
attracting the detection sample with a first magnetic element to the light-transmitting groove;
displacing the plate on the substrate laterally so that the light-transmitting groove is on top of the hole of the substrate; and
irradiating the detection sample by a first light of a light source, so that the light conversion materials in the detection sample converts the first light to a second light, which is absorbed by a photoelectric conversion element to produce a current.

2. The method of claim 1, wherein the test sample comprises a specimen and a chemical that is the protein kinase.

3. The method of claim 2, wherein in the step in which the test sample is heated to the first temperature, a heating element heats the substrate to a first temperature; the first temperature is over 50° C. and under 100° C.

4. The method of claim 1, wherein after the magnetic nanoparticles are adsorbed onto the test sample, a second magnetic element magnetically attracts the test sample to the bottom of the groove, on which magnetic nanoparticles are adsorbed.

5. The method of claim 4, wherein in the step in which the second magnetic element attracts the test sample on which magnetic nanoparticles are adsorbed to the bottom of the groove, further comprising the steps:
continuously inject a cleaning solution, via the first guide hole into the groove, to remove the test sample that does not have magnetic nanoparticles adsorbed; and
the cleaning solution flows via the first drainage hole on the side of the first guide hole to the waste liquid reservoir element.

6. The method of claim 1, wherein the light conversion materials are injected via the first injection hole and the first guide hole into the groove to produce a detection sample, further comprising the steps:
continuously inject a cleaning solution, such that it flows via the first guide hole into the groove to remove light conversion materials not adsorbed to the test sample; and
the cleaning solution flows via a first drainage hole on the side of the first guide hole to a waste liquid reservoir element.

7. The method of claim 5, wherein the cleaning solution is ethanol or acetone.

8. The method of claim 6, wherein the cleaning solution is ethanol or acetone.

9. The method of claim 1, wherein the test sample is cooled to the second temperature, the test sample is cooled by a cooling line containing a coolant that surrounds the groove, and the second temperature is between 20° C. and 30° C.

10. The method of claim 1, wherein the wavelength of the light source is from 200 nm to 700 nm.

11. The method of claim 1, wherein the light conversion materials are fluorescein, phosphor, or quantum dots.

12. The method of claim 1, wherein the detection sample is irradiated by the first light from the light source through the hole, the detection sample absorbs the first light and converts it to the second light by the light conversion materials in the detection sample, the photoelectric conversion element absorbs the second light and produces a current, and further comprising the steps:
A filter the first light not converted in the detection sample by a light filter element, while the second light passes through a light filter and light-transmitting element and be absorbed by the photoelectric conversion element.

13. A configuration of the nucleic acid detection chip comprising:
a sliding chip element, which has a plate and a substrate, wherein the substrate includes the first body, a groove, and a hole, and the plate includes the second body, the first injection hole, and a light-transmitting groove, and the plate slide is on the substrate, the groove is on the top of the first body, the first injection hole is connected to the first guide hole, the light transmitting groove is on the side of the first injection hole; and
a photoelectric conversion element is on the light-transmitting groove;
wherein the groove contains a detection sample, which has the test sample with magnetic nanoparticles and light conversion materials adsorbed on; and through sliding, the plate causes the light-transmitting groove to be above the groove and attracts the detection sample to the light-transmitting groove by the first magnetic element, and through sliding, the plate causes the light-transmitting groove to be displaced above the hole and allows the first light of the light source to irradiate the detection sample, and he light conversion materials convert the first light to the second light, and the photoelectric element absorbs the second light and produces a current.

14. The configuration of claim 13, wherein the test sample is injected into the groove via the first injection hole and first guide hole, the substrate is heated by a heating element to the first temperature and cooled to the second temperature; the magnetic nanoparticles are injected via the first injection hole and the first guide hole to the groove and adsorbed to the test sample; the light conversion materials are injected into the groove via the first injection hole and the first guide hole, and adsorbed to the test sample to produce a detection sample, and the test sample includes a specimen and a chemical that is the protein kinase, and the first temperature is over 50° C. and under 100° C., and the second temperature is between 20° C. and 30° C.

15. The configuration of claim 13, comprising of:
a cooling line containing coolant that surrounds the groove;
a light filter element above the light-transmitting groove; and
a light-transmitting element above the light filter element.

16. The configuration of claim 13, wherein the wavelength of the light is from 200 nm to 700 nm, and the light conversion materials are fluorescein, phosphor, or quantum dots, which are used to convert the wavelength of the light.

17. The configuration of claim 13, wherein the first injection hole has a first diameter, and the first guide hole has a second diameter, and the first diameter is bigger than the second diameter.

18. Apparatuses of the nucleic acid detection chip, comprising:
a carry base, on which there is a light source;
a sliding chip element, disposed on the carry base that has a substrate that has the first body, a groove, and a hole, and a plate that has the second body, the first injection hole, and the light-transmitting groove; the plate slide is on the substrate, the groove is on the first body, the first injection hole is connected to the first guide hole, and the light-transmitting groove is on the side of the first injection hole;
a photoelectric conversion element, its on the light-transmitting groove;
a moving element, includes a moving part and an electrode sensing part, and a display element that is electrically connected to the electrode sensing part, and a moving part, disposed on the side of the plate, and the electrode sensing part is electrically connected to the photoelectric conversion element; and
a display element, its electrically connected to the electrode sensing part;
wherein, the groove contains a detection sample, which has the test sample with magnetic nanoparticles and light-conversion materials adsorbed on, and through sliding the plate by the moving part, the light-transmitting groove is displaced on the groove and attracts the detection sample to the light-transmitting groove with the first magnetic element, and Through sliding the plate, the light-transmitting groove is displaced on the hole and allows the first light of the light source to irradiate the detection sample, And the light conversion material converts the first light to the second light, and the photoelectric element absorbs the second light and produces a current, which is received by the electrode sensing part; the result from the test is then reflected on the display element.

19. A cleaning method of the nucleic acid detection chip, which is applied after the test sample is injected into the first injection hole on the plate and causing it to flow via the first guide hole into the groove on the substrate; injecting magnetic nanoparticles via the first guide hole into the groove to mix with and adsorb to the test sample; injecting light conversion materials into the groove to produce a detection sample; displacing the plate laterally so that the test hole is on the groove, and attracting the detection sample using the first magnetic element to the light-transmitting groove; displacing the plate laterally so that the detection sample in the light-transmitting groove is on the hole of the substrate; and irradiating the detection sample by the first light of the light source through the hole so that it absorbs the first light and converts it, using the light conversion materials in the detection sample, to the second light, which is absorbed by the photoelectric conversion element to produce a current, and the steps in the method for the nucleic acid detection chip's cleaning method comprise the steps:
displace the plate laterally so that the light-transmitting groove on the plate is on the groove of the substrate;
inject cleaning solution via the cleaning channel on the side of the light-transmitting groove into the groove to remove the detection sample in the light-transmitting groove and the groove; and
the cleaning solution will flow via the second drainage hole on the side of the light-transmitting groove to the waste liquid reservoir element.

20. A cleaning device for the nucleic acid detection chip, comprising:
a substrate, which comprises a first body, a groove, and a hole, with the groove on the body;
a plate, which comprises a second body, and the second body with a first injection hole and a light-transmitting groove, and the first injection hole is connected to the first guide hole, and the light-transmitting groove is on the side of the first injection hole and the first injection hole has the first diameter, and the first guide hole has the second diameter, and the first diameter is bigger than the second diameter, and there is a drainage hole beside the first guide hole and a second drainage hole between the first guide hole and the light-transmitting groove; and
a photoelectric conversion element, disposed on the plate;
Wherein the plate is displaced laterally so that the light-transmitting groove is on the groove on the substrate, and cleaning solution can be injected into the light-transmitting groove, and the cleaning solution flows via the light-transmitting groove into the groove to remove the detection sample in the light-transmitting groove and groove, the cleaning solution flows via the drainage hole on the side of the light-transmitting groove to the waste liquid reservoir element.

21. The cleaning device of claim 20, wherein there is a heating element on the side of the substrate that heats the substrate to the first temperature, and the first temperature is over 50° C. and under 100° C.

22. The cleaning device of claim 20, further comprising:
a cooling line, surrounding the groove, which delivers a coolant;
a light filter element, disposed on the light-transmitting groove; and
a light-transmitting element, disposed on the light filter element.

23. The cleaning device of claim 20, wherein the first injection hole has the first diameter, and the first guide hole has the second diameter, and the first diameter is bigger than the second diameter.
